(19) United States
(12) Patent Application Publication
(10) Pub. No.: US 2007/0237295 A1
(43) Pub. Date: Oct. 11, 2007

(54) Tomography system and method for visualizing a tomographic display

(76) Inventor: Lutz Gundel, Erlangen (DE)

Correspondence Address: HARNESS, DICKEY & PIERCE, P.L.C., P.O.BOX 8910, RESTON, VA, 20195, US

(21) Appl. No.: 11/657,020

(22) Filed: Jan. 24, 2007

(30) Foreign Application Priority Data
Jan. 25, 2006 (DE) 10 2006 003 609.3

(51) Int. Cl. G01N 23/04; A61B 5/05

(52) U.S. Cl. 378/62; 600/425

(57) ABSTRACT

A tomography system and a method are disclosed for visualizing a tomographic display. The system, in at least one embodiment, includes a detector system for scanning an object, an arithmetic logic unit for conditioning the data determined by the scanning, and a device for visualizing tomographic image data of the scanned object with a first image resolution. According to at least one embodiment of the invention, the device for visualizing the tomographic image data has a selectable display function by which a subregion of the visualization is marked, and this marked subregion is simultaneously displayed in a second, relatively higher image resolution in addition to the visualization in the first image resolution, the calculation of the tomographic display being performed with the first, relatively low resolution, and a recalculation with the set, relatively higher resolution being carried out for the marked subregion.

### PRIORITY STATEMENT

The present application hereby claims priority under 35 U.S.C. §119 on German patent application number DE 10 2006 003 609.3 filed Jan. 25, 2006, the entire contents of which is hereby incorporated herein by reference.

### FIELD

Embodiments of the invention generally relate to a tomography system. For example, they may relate to one including a detector system for scanning an object, in particular a patient, an arithmetic logic unit for conditioning the data determined by the scanning, and a device for visualizing tomographic image data of the scanned object with a first image resolution. Moreover, embodiments of the invention also generally relate to a method for visualizing a tomographic display in a tomography system. For example, they may relate to one in which an object, in particular a patient, is scanned with at least one detector system, tomographic object data with a tomographic resolution are calculated with the aid of the data determined by the scanning, and the tomographic object data are displayed as a sectional image with a first resolution.

### BACKGROUND

It is presently customary when displaying topographic images for the resolution and the enlargement on the display screen resulting therefrom to be selected variably. The image matrix used for the display generally has 512×512 pixels. If an overview of a sectional image of a patient is displayed here, the resolution displayed is substantially lower than the technically possible resolution of the detector system being used, particularly when use is being made of a CT system. If the user now selects a higher resolution in order to be able to better detect details in the image, the overview is very quickly lost and there is at least a need to switch very inconveniently to and fro between the overview resolution and the detail resolution.

### SUMMARY

In at least one embodiment of the invention, a tomography system and method are disclosed for visualizing a tomographic display that enable details to be displayed in a more effectively detectable fashion without restricting the overview.

The inventor has found, in at least one embodiment, that a combined display of an overview image and a detail enlargement improves upon or even solves the problem of orientation and, at the same time, enables the improved resolution and enlarged display required for assessing a specific image region, doing so by a type of close-up display of a selected region. In this case, the user can determine which region in an overview image he would like to see as a detailed display, and in which resolution, and thus in which enlargement, he would like this detail region to be displayed. It is conducive to ease of handling in this case when, for example with the aid of a positioning device, for example a mouse or a trackball, the region to be enlarged can be displaced at will on the overview image, and this region simultaneously displayed in the higher resolution display, as seen through a magnifying glass.

It is fundamentally possible with this mode of display firstly to reconstruct or to calculate the entire region to be displayed in the maximum possible resolution, to show the overview display only in a reduced resolution, and at the same time, once again, to show the “magnifying glass region” in the desired to maximum resolution. The disadvantage of this variant lies in the fact that a high outlay on computation and storage is required to calculate the display for the first time, although only a low computational outlay is required during the display itself when displacing the region to be enlarged or changing the resolution. In another variant, the overview image can be calculated in a low resolution, and it is not until after the desired detail enlargement and/or the desired detail region have been defined that the desired and, if appropriate, maximum resolution is recalculated. These variants relate not only to the calculation of primary display data, but also to additional variations of the display, for example by volume rendering, segmenting and the like.

In accordance with the basic idea of at least one embodiment of the invention outlined above, the inventor proposes a tomography system that has at least one detector system for scanning an object, in particular a patient, an arithmetic logic unit for conditioning the data determined by the scanning, and a device for visualizing tomographic image data of the scanned object with a first image resolution. For the purpose of improvement, the device for visualizing the tomographic image data is to have a selectable display function by which a subregion of the visualization is marked, and this marked subregion is simultaneously displayed in a second, higher image resolution in addition to the visualization in the first image resolution.

Here, the selectable display function can reproduce the visualization in the second, higher image resolution as image in image of the first image resolution. It is possible, in addition, that the selectable display function reproduces the display in the second, higher image resolution at a prescribed minimum distance or a maximum possible distance from the marked subregion. The result of this is that the enlarged subregion being viewed is as far as possible free from being covered by the enlarged display itself.

Alternatively, the tomography system can also be equipped for visualization with a further display such that the visualization with the first resolution can be displayed on the first display, and the visualization with the higher resolution can be displayed on the second display.

It is also advantageous when provided for the image region that is marked and to be enlarged is a positioning device by means of which a user can arbitrarily displace this marked region on the pictorial display with the first resolution. This can be, for example, a mouse, a trackball or else a touch-sensitive display screen.

It can also be advantageous for an improved optical guidance of the user to produce an optical connection between the image region, which is marked and to be enlarged, with the first resolution and the image region displayed with higher resolution. This can be performed, for example, by displaying linear connecting lines between the marked region and the region displayed in an enlarged fashion.

It is also possible to provide a function for the user to directly select the first and/or the second resolution. For example, this can be done by direct numerical input, or a potentiometer.

The invention, in at least one embodiment, also proposes a method for visualizing a tomographic display in a tomography system, it being known that the latter scans an object, in particular a patient with at least one detector system, calculates tomographic object data with a tomographic resolution with the aid of the data determined by the scanning, and displays the tomographic object data as a sectional image with a first resolution. According to the invention, in this case a display function is made available by which a subregion of the visualization can be marked and this marked subregion can be simultaneously displayed in at least one second, higher image resolution in addition to the visualization in the first image resolution.

In accordance with at least one embodiment of the previously described tomography system, the visualization can be performed with the at least one second, higher image resolution as image in image of the first image resolution. It is also possible here for the display with the at least one second, higher image resolution to be placed such that it is displayed at a prescribed minimum distance or a maximum possible distance from the marked subregion.

Alternatively, two displays can be used for the visualization, and the display with the first resolution can be performed on the first display, and the display with the higher resolution can be performed on the second display.

It is, furthermore, helpful when the image region that is marked and to be enlarged can be displaced by a cursor movement in the first display. Moreover, the user can determine the extent of the marked region on the pictorial display by way of a cursor movement.

It is also advantageous for at least one embodiment of the method to be configured such that the user can vary the extent and/or position of the at least one display with higher resolution.

In order to fashion the use of the tomographic system as intuitively as possible, the displayed higher resolution can be determined automatically on the basis of the ratio of dimensions between the marked region in the first resolution, and the selected display size in the at least one second resolution. There is thus no need for knowledge relating to the resolution used, and the user obtains his desired enlargement and/or resolution by simply defining the size of the “magnifying glass”.

It is also advantageous for a better overview of the display when an optical connection is produced for example by lines added in color between the image region, which is marked and to be enlarged, with the first resolution and the image region displayed with higher resolution.

The user can also be offered a function for directly selecting the first and/or the second resolution.

In a particular variant of at least one embodiment of the invention, the inventor proposes that the tomographic display be calculated with the maximum possible resolution, and the data thereof be stored, that the first display with reduced resolution be produced from these stored data, and that the region with higher resolution likewise be obtained therefrom. As already mentioned above, this mode of procedure firstly requires a relatively high arithmetic capability, but after the calculation of the display with an optimum resolution a display with different, lower resolutions can be performed by way of relatively simple computing steps. The displacement and simultaneously enlarged display of the marked region can be performed correspondingly quickly.

Alternatively, the tomographic display can firstly be calculated with a first, low resolution, and a recalculation can be carried out for the region with higher resolution in accordance with the higher resolution set. A very quick first overview display is enabled thereby, the display of the enlargement requiring a corresponding time outlay.

It is also possible for the overview image to be calculated quickly with low resolution and displayed immediately. As the user now considers this low resolution display, orients himself and clarifies which regions of the image are of particular interest to him, a high resolution calculation can already be performed in parallel with the aid of the free arithmetic capability during the waiting time, such that this high resolution display is available at once as soon as the user requests a partial view thereof. In this case, the user is very quickly provided with the overview, and the delay for the high resolution display is scarcely detectable.

It can, moreover, be advantageous when a resolution that can be achieved with the given detector system is used as maximum settable, higher resolution, or an optical or acoustic indication is triggered at least when a resolution higher than that which can be achieved with the given detector system is set.

At least one embodiment of the above described method can be used, in particular, in conjunction with a CT system, it being possible for primary reconstructed tomographic sectional images to be displayed simultaneously with different resolutions.

However, at least one embodiment of this method is also advantageous in conjunction with the display of so-called secondary reconstructions. These are, for example, a “multiplanar reconstruction” (MPR), a “maximum intention projection” (MIP), a “volume rendering technique” (VRT) or a “surface shaded display” (SSD). This enumeration is not definitive.

In addition to the use of the described method in the field of CT, it is also possible to use at least one embodiment of the method in conjunction with a PET system, an NMR system or an ultrasound system.

Without departing from the scope of the invention, the inventor also proposes, in at least one embodiment, a storage medium integrated in an arithmetic logic unit or for an arithmetic logic unit of a tomography system that includes at least one computer program or program modules stored thereon, that, at least partially, executes the above-described method, when executed on the arithmetic logic unit of the tomography system.

### BRIEF DESCRIPTION OF THE DRAWINGS

The invention is explained in more detail below with reference to a an example embodiment and with the aid of the figures, only the features required to understand the invention being illustrated. The following reference symbols have been used to describe the figures: 1: CT system; 2: X-ray tube; 3: detector; 4: gantry opening; 5: control and data line; 6: gantry housing; 7: patient; 8: patient couch; 9: system axis/z-axis; 10: arithmetic logic unit; 11: storage medium; 12: MPR image; B: marked image region; B+: marked image region displayed with high resolution; Prgₓ: computer programs. In detail:

FIG. 1 shows a computed tomography system, and

FIG. 2 shows an example of an inventive display of a CT image.

### DETAILED DESCRIPTION OF THE EXAMPLE EMBODIMENTS

It will be understood that if an element or layer is referred to as being “on”, “against”, “connected to”, or “coupled to” another element or layer, then it can be directly on, against, connected or coupled to the other element or layer, or intervening elements or layers may be present. In contrast, if an element is referred to as being “directly on”, “directly connected to”, or “directly coupled to” another element or layer, then there are no intervening elements or layers present. Like numbers refer to like elements throughout. As used herein, the term “and/or” includes any and all combinations of one or more of the associated listed items.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

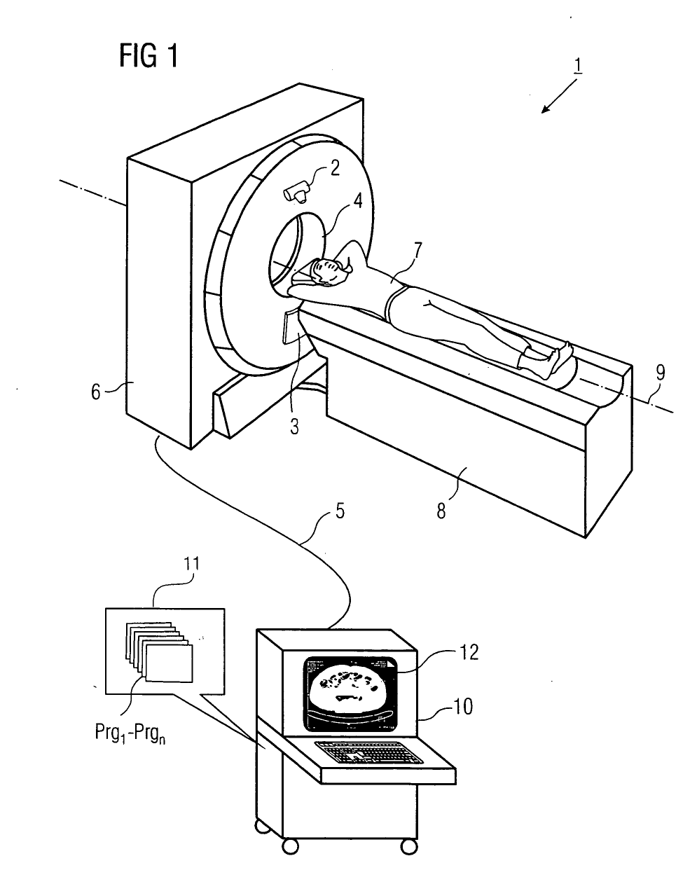

FIG. 1 shows by way of example a computed tomography system 1 in which an embodiment of the invention presented has been implemented. The computed tomography system 1 is known to have a gantry housing 6 (with a gantry not shown in detail) on which an X-ray tube 2 and an opposing detector 3 are fastened. Located between the X-ray tube 2 and the detector 3 is a scanning opening 4 through which a patient 7 to be scanned can be transported with the aid of a patient couch 8 that can be displaced along the system axis or z-axis 9, while the X-ray tube 2 and the detector 3 rotate about the system axis 9 and in this way scan the patient 7 spirally or, in a stepwise fashion, circularly—relative to the coordinate system of the patient 7.

The attenuation determined in the X-ray beams upon passage through the patient is determined by the detector 3 and passed on, via a control and data line 5, to the arithmetic logic unit 10. There, the computer programs Prgₓ, which are stored in a storage medium 11 (indicated merely schematically) and can be retrieved in case of need from the main memory of the CPU of the arithmetic logic unit, are used to condition and, mostly, to resort the data on parallel projections, and a volumetric display of the absorption properties of the scanned region is reconstructed by means of known methods. Such a volumetric display can be a multiplicity of sectional images arranged sequentially in the direction of the system axis, or the voxels of the scanned region are actually reconstructed individually. Sectional images in any desired planes can, in turn, be extracted from the multiplicity of voxels now known for the purpose of visualization on a two-dimensional display screen.

Furthermore, it is possible to select additional display or conditioning variants by which, for example, a segmenting, a so-called “volume rendering”, or another similar method that facilitates the assessment of the images is carried out.

It is possible on the basis of the physical properties of the detector and the scanning undertaken to achieve specific maximum resolutions that cannot, however, be visualized with the aid of conventional display screens in the case of an overview display of a scanned region. The pixel number of normal display screens is not sufficient to this end, and a display screen with an adequate pixel number would be much too large and thus would also not enable the user to work effectively.

The inventor therefore proposes to select a parallel display with different resolutions in one embodiment, in which case, for example, the overview is visualized with low resolution on the same display screen, a region to be displayed more effectively is selected there, and this selected region is shown in an overlapping display with higher resolution.

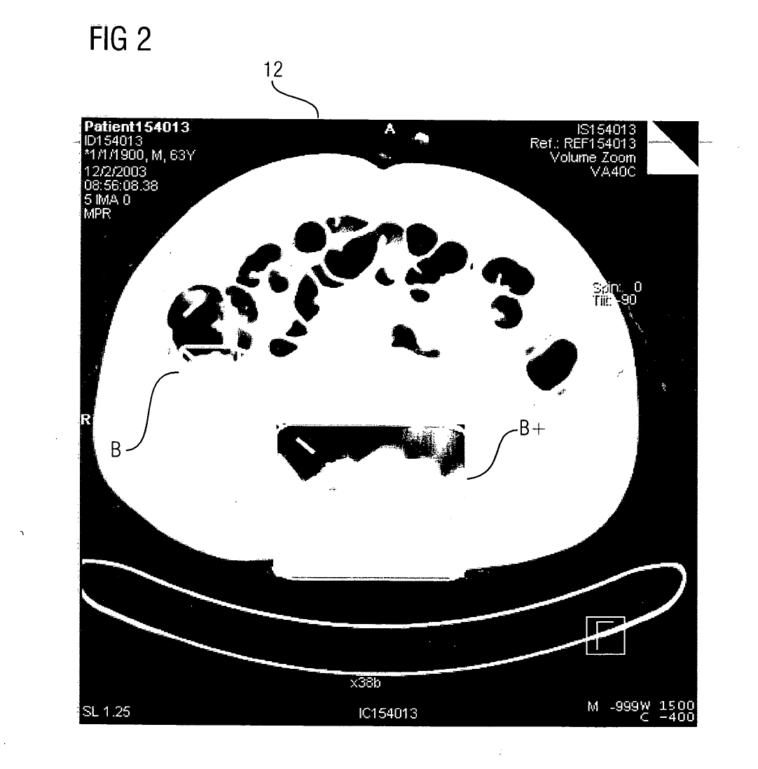

FIG. 2 shows such a display of an MPR image 12 with a CT of an abdomen. Marked in this display is a region B of the colon that is classified as suspicious by the viewer and is thereupon displayed automatically in an overlaid “window” B+ with the maximum possible resolution in a fashion offset from the marked region B. In addition, connecting lines are displayed between the four corners of the two regions B and B+ for the purpose of easier orientation.

The viewer now detects in the enlarged display of higher resolution that the region of the colon classified as suspicious in the overview image is, because of the air inclusions now to be detected, actually only a residual stool and not a malignant tissue. The user can now be given the possibility of making the marked region migrate over the display screen by “clicking” the marked region B and subsequently moving a pointer, for example of a mouse or a trackball, and of viewing the suspicious sites of the overview image with a magnifying glass, as it were, without losing his orientation in a large image that cannot be visualized completely on the display screen, and of inspecting important regions, if appropriate.

It remains to be remarked by way of supplement that not only is it possible to displace the marked region in the plane of the visualization of the sectional image, but that, the overall view with the marked region B and the more precise display B+ can also be moved in the z-direction, for example by actuating the mouse wheel. Since the position of the marked region in the image plane is simultaneously determined via the pointer, a very simple virtual movement, or a type of virtual flight in the three-dimensional scanned volume is possible in this way, the marked region B+ simultaneously always remaining displayed in an enlarged fashion. The orientation is thereby very simple. It is not only the displacement of the enlargement region that is advantageous here, but also its interactive enlargement and reduction.

Equally capable of implementation is the volume rendering of pulmonary lesions, in which case the above-described high resolution lens can, for example, indicate the supply of small vessels that is important for diagnosis and subsequent therapy. The volume measurement of lesions is of greater importance in the field of oncology. Here, the resolution of the data voxels is an important parameter that determines their accuracy. The use of high resolution voxels leads here to a clearly more accurate measurement.

Two different computational variants of implementation are possible here in principle. Firstly, the complete data record can be reconstructed twice, with normal and with maximum accuracy. By way of suitable overlaying, the “high resolution voxels” can then be fed to the visualization, or to the further processing in a secondary reconstruction and to the subsequent visualization. On the other hand, it is possible for only precisely the required volume to be reconstructed anew with high resolution and to serve as input data for the secondary reconstruction and/or the visualization.

It goes without saying that the features of the invention mentioned above can be used not only in the combination respectively specified, but also in other combinations, or on their own without departing from the scope of the invention.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A tomography system, comprising:
a detector system to scan an object;
an arithmetic logic unit to condition data determined by the scanning;
means for visualizing tomographic image data of the scanned object with a first image resolution, including a selectable display function by which a subregion of the visualization is marked, the marked subregion being simultaneously displayed in a second, relatively higher image resolution in addition to the visualization in the first image resolution, a calculation of the tomographic display being performed with the first, relatively low resolution, and a recalculation with the set, relatively higher resolution being carried out for the marked subregion.

2. The tomography system as claimed in claim 1, wherein the selectable display function reproduces the visualization in the second, relatively higher image resolution as image in image of the first image resolution.

3. The tomography system as claimed in claim 2, wherein the selectable display function reproduces the display in the second, relatively higher image resolution at a prescribed minimum distance or a maximum possible distance from the marked subregion.

4. The tomography system as claimed in claim 1, wherein the means for visualizing includes two displays, and the visualization with the first resolution is displayed on the first display, and the visualization with the relatively higher resolution is displayed on the second display.

5. The tomography system as claimed in claim 1, further comprising, for the image region that is marked and to be enlarged, a positioning device by which a user can arbitrarily displace this marked region on the pictorial display with the first resolution.

6. The tomography system as claimed claim 1, wherein an optical connection is produced between the image region, which is marked and to be enlarged, with the first resolution and the image region displayed with relatively higher resolution.

7. The tomography system as claimed in claim 1, wherein a function for the user to select at least one of the first and the second resolution is provided.

8. The tomography system as claimed in claim 1, wherein the system is a CT system.

9. The tomography system as claimed in claim 1, wherein the system is a PET system.

10. The tomography system as claimed in claim 1, wherein the system is an NMR system.

11. A method for visualizing a tomographic display in a tomography system, comprising:
scanning an object with at least one detector system;
calculating tomographic object data with a tomographic resolution with the aid of data determined by the scanning; and
displaying the tomographic object data as a sectional image with a first resolution, wherein a display function is made available by which a subregion of the visualization is markable and this marked subregion is simultaneously displayable in at least one second, relatively higher image resolution in addition to the visualization in the first image resolution, the calculation of the tomographic display being performed with the first, relatively low resolution, and a recalculation according to the set, relatively higher resolution being performed for the region with relatively higher resolution.

12. The method as claimed in claim 11, wherein the selectable display function reproduces the visualization in the at least one second, relatively higher image resolution as image in image of the first image resolution.

13. The method as claimed in claim 12, wherein the display in the at least one second, relatively higher image resolution is displayed at at least one of a minimum distance and a maximum possible distance from the marked subregion.

14. The method as claimed in claim 11, wherein the means for visualizing includes two displays, and the display with the first resolution is displayed on the first display, and the display with the relatively higher resolution is displayed on the second display.

15. The method as claimed in claim 11, wherein the image region that is marked and to be enlarged is displaceable by a cursor movement in the first display.

16. The method as claimed in claim 11, wherein, via a cursor movement, the extent of the marked region on the pictorial display is determinable.

17. The method as claimed in claim 11, wherein at least one of the extent and position of the at least one display with relatively higher resolution is variable.

18. The method as claimed in claim 11, wherein the displayed relatively higher resolution is determined automatically on the basis of the ratio of dimensions between the marked region in the first resolution, and the selected display size in the at least one second resolution.

19. The method as claimed in claim 11, wherein an optical connection is produced between the image region, which is marked and to be enlarged, with the first resolution and the image region displayed with relatively higher resolution.

20. The method as claimed in claim 11, wherein a function for selecting at least one of the first and the second resolution is offered to a user.

21. The method as claimed in claim 11, wherein a resolution that is achievable with the given detector system is used as maximum settable relatively higher resolution.

22. The method as claimed in claim 11, wherein at least one of an optical and acoustic indication is triggered when a resolution higher than that which is achievable with the given detector system is set.

23. The method as claimed in claim 11, wherein a CT system is used.

24. The method as claimed in claim 23, wherein primary reconstructed tomographic sectional images are displayed.

25. The method as claimed in claim 23, wherein tomographic sectional images from a “multiplanar reconstruction” (MPR) are displayed.

26. The method as claimed in claim 23, wherein tomographic sectional images from a “maximum intention projection” (MIP) are displayed.

27. The method as claimed in claim 23, wherein tomographic sectional images from a “volume rendering technique” (VRT) are displayed.

28. The method as claimed in claim 23, wherein tomographic sectional images are displayed as a “surface shaded display” (SSD).

29. The method as claimed in claim 11, wherein a PET system is used.

30. The method as claimed in claim 11, wherein an NMR system is used.

31. The method as claimed in claim 11, wherein an ultrasound system is used.

32. A storage medium at least one of integrated in an arithmetic logic unit and for an arithmetic logic unit of a tomography system, including at least one computer program or program module that, when executed on the arithmetic logic unit of the tomography system, executes the method as claimed in claim 11.

33. The tomography system as claimed claim 3, wherein an optical connection is produced between the image region, which is marked and to be enlarged, with the first resolution and the image region displayed with relatively higher resolution.

34. The tomography system as claimed claim 5, wherein an optical connection is produced between the image region, which is marked and to be enlarged, with the first resolution and the image region displayed with relatively higher resolution.
